(19) United States
(12) Patent Application Publication
(10) Pub. No.: US 2021/0326939 A1
(43) Pub. Date: Oct. 21, 2021

(54) SOCIAL PAYMENT FOR MEDICAL BILLS AND BILL SETTLEMENT

(71) Applicant: Murgesh Navar, San Jose, CA (US)

(72) Inventor: Murgesh Navar, San Jose, CA (US)

(21) Appl. No.: 17/230,657

(22) Filed: Apr. 14, 2021

Related U.S. Application Data
(60) Provisional application No. 63/011,188, filed on Apr. 16, 2020.

(51) Int. Cl. G06Q 30/02; G06Q 30/04; G06Q 20/14; G06Q 20/40; G16H 40/00

(52) U.S. Cl. CPC G06Q 30/0279; G06Q 30/04; G16H 40/00; G06Q 20/4016; G06Q 20/145

(57) ABSTRACT

The disclosure provided herein is directed to a payment system configured to aggregate one or more bills related to one or more items in an online shopping environment or for one or more services or products already provided to a consumer or expected to be provided to a consumer in a single invoice or super-invoice, which in some instances is reviewed for consistency, errors, and gaps in service or products. Based on the super-invoice, the payment system is configured to counter the amount owed as well as collect payment for one or more benefactors through a variety of social media and/or networks to satisfy all or some of the amount owed. To facilitate the sufficiency of payment, the payment system is further configured to determine a consumer affordability score that corresponds to a consumer's capability to pay an amount owed.

### CROSS REFERENCE TO RELATED APPLICATION

This application claims the benefit of priority from U.S. Provisional Patent Application Ser. No. 63/011,118 filed Apr. 16, 2020, the contents of this application is hereby incorporated by reference in its entirety.

### BACKGROUND OF THE INVENTION

This disclosure generally relates to a method and system for social payment of medical bills and medical settlement. More specifically, and without limitation, this disclosure relates to a method and system for social payment of medical bills and medical settlement based on a comparison of going rates, identifying billing errors, and providing an affordability score related to the payee of the bill for the bill collector to then use when determining whether they will accept an offer of settlement provided by that payee. The following background and portions of the disclosure relate specifically to healthcare costs and medical bills for illustrative purposes only, but it is anticipated that this invention may be applied in various other service provider configurations and bill payment systems outside of healthcare providers.

Bill payment systems are well known in the art. The overall cost of healthcare has increased substantially in recent times making quality healthcare unaffordable to millions of people across the globe. Insurance companies pay a medical bill claim only if the consumer has purchased sufficient healthcare insurance and such health insurance is active at the time of a medical need. With U.S. healthcare costs far outpacing income growth, and higher patient cost-sharing through deductibles, co-payments, and co-insurance, consumers are being asked to pay a substantial cost of a medical bill out of their own pocket before and after their insurance kicks in.

137.1 million Americans faced financial hardship in 2019 because of medical costs and two-thirds of all personal bankruptcies were tied to medical bills. Unaffordable healthcare is increasingly a global phenomenon. According to a recent report by the World Economic Forum, the unaffordability of healthcare pushes 100 million people into poverty each year. Another 179 million people globally spend 25% of their total household income on medical costs.

For U.S. consumers with some form of medical insurance, the billing process begins when the provider files claims with the consumer's insurer for the cost of treatment. Once the insurer determines the amount of reimbursement, the provider then bills the consumer for the remaining balance subject to any limitations that may have been agreed upon between the provider and insurer. Most consumers with medical insurance coverage are still responsible for some portion of the billed cost of care before or after obtaining medical treatment, until an annual out-of-pocket maximum on consumer payments is reached. Consumer obligations can take the form of co-payments, the cost of drug prescriptions, or annual per-person and/or per-household deductible amounts the consumer must pay before insurance is paid out. These factors can determine a consumer's share of out-of-pocket expenses for a particular treatment. Those without health insurance are responsible for paying providers for the unnegotiated full list price of the healthcare they receive.

Many consumers borrow from personal lenders to pay their medical debt. However, the availability of such funds is highly dependent on the credit score of the consumer. When a U.S. person can't pay a medical bill, that debt is often packaged with other people's medical debt and sold to bill collectors for a small fraction of the total amount of the bill, averaging $0.04 for each $1 dollar owed. When a bill ends up with debt collectors, the consumer experiences a negative impact on their credit score resulting in a higher cost of borrowing credit when getting a car loan, mortgage, or credit card. Unable to face the people they owe, many patients also forego follow-up treatment.

Alternatively, many consumers borrow money from friends, family, and other patrons to settle their medical bills. However, such social borrowing is a tedious, at times embarrassing, and manual process that is not readily accomplish. Many consumers also turn to crowdfunding sites to solicit donations to pay for their medical expenses—often from strangers. However, without assurances of the legitimacy of the need or the genuine use of the donation proceeds, many potential patrons are reluctant to help.

As patient responsibility has increased from 10% to 30% in recent years, recovering out-of-pocket costs has become increasingly more critical for U.S. hospitals. According to some estimates, out-of-pocket costs in the U.S. will increased from $250B to $420B in the next few years. Many healthcare providers are suing patients to recover their dues, adding further expenses associated with such litigation. When healthcare service providers are not paid fairly for the services they provide, it discourages them from providing quality health services for millions of needy people.

A major cause for disputes between the services provider and the consumer is due to mistakes made by the service provider while preparing the medical bills. Some estimates show that 30% to 40% of medical bills contain errors, with hospital bills that totaled more than $10,000 containing an average error of $1,300. In many cases the charges billed by the service provider are excessive in comparison to customary charges, while in other cases there are errors in medical bills due to a typo, a duplicate entry, or an incorrect medical code entry. In some cases, even fraudulent entries are made for services that were never provided. In some of these cases, the overall cost associated with the medical claim is not acceptable to the consumer leading to disputes and non-payment of the bill. The efficient settlement of such bills favors the service provider by saving precious time and resources spent in the recovery of the pending medical bills, and continued business by preserving the doctor-patient relationship.

It is general practice across the globe to seek financial help from friends, family and patrons at the time of medical need. According to an estimate, 37% of U.S. healthcare consumers borrow from friends, family and patrons to settle their medical bills.

The present system overcomes the aforementioned deficiencies faced by both the consumers and the service provides, and aids in a faster recovery of pending bills while helping assure consumers of a fairly priced bill, thus preserving the doctor-patient relationship.

Thus it is a primary aspect of this invention to provide a method and system for social payment of medical bills and medical settlement that improves upon the prior art.

Another aspect of this disclosure is to allow a consumer to upload one or more bills received from one or more service providers, including healthcare service providers.

Yet another aspect of this disclosure is to utilize big data, artificial intelligence, and deep learning technologies to extract and identify billing errors, including incorrect pricing, coding errors, and the like.

Another aspect of this disclosure is to provide a comparison of service prices offered by other service providers in the vicinity.

Yet another aspect of this disclosure is to generate a super-invoice by aggregating multiple bills, including when multiple service providers are involved.

Another aspect of this disclosure is to provide direct online payment, partial or complete, by the consumer, friends, family, and patrons thereof.

Yet another aspect of this disclosure is to share a super-invoice with friends, family, and patrons for a contribution towards the payment of the super-invoice.

Another aspect of this disclosure is to generate a consumer affordability score based on personal and social information of the consumer which is an indicator of the ability of the consumer to pay.

Another aspect of this disclosure is to utilize artificial intelligence and deep learning to rectify billing errors, inconsistencies, and overcharges.

Yet another aspect of this disclosure is to maximize recovery of amounts owed by consumers without the need for repeated manual follow-ups, bill collection actions, or litigation.

Another aspect of this disclosure is to guide service providers to accept or reject a proposed settlement offer based on the consumer affordability score.

Yet another aspect of this disclosure is to provide a system that identifies items of needed care not provided or sought by the consumer for a specific diagnosis.

These and other aspects, features, and advantages of the disclosure will become apparent from the specification and claims.

### SUMMARY OF THE INVENTION

The disclosure provides various aspects of a method and system for social payment of medical bills and medical settlement. In one aspect of the disclosure, broadly described herein, a method or system is described that aggregates one or more bills for services and goods that have been provided into a super-invoice, wherein a variety of billing errors and consistencies are identified through comparative analysis. In one aspect, funding for the super-invoice is obtained through one or more benefactors, which are thereafter applied to pay the entire or a portion of the super-invoice. In another aspect of the disclosure, partial payment or payment of a revised amount owed based on the identified inconsistencies and errors is provided to the one or more service providers. In one aspect, a consumer affordability score is provided to the one or more service providers to determine whether the offer of payment aligns with the capability to pay. In other aspects of the present disclosure, broadly described herein, the above system and method is applied prior to the purchase of goods or services, such as in instances having recurring payments or in instances involving online shopping for one or more product and/or service.

In one aspect of the disclosure, the payment system is provided to service providers to directly transmit digital invoices. In another aspect, the payment system is provided to receive digital images of paper invoices received by a consumer. In an aspect of the present disclosure, data is extracted from the invoices, including by way of OCR algorithms, which is thereafter presented to the consumer for payment, including by way of a direct payment button on the payment system and/or a separate digital invoice transmitted to the consumer by way of electronic means that includes a URL to a digital invoice.

In one aspect, the payment system aggregates multiple invoices pertaining to a single episode presents the itemized bill in a single super-invoice providing an aggregate total amount owed. In another aspect, broadly described herein, the payment system is configured during a training phase to identify fraudulent and abusive itemizations as well as entries commonly presented in similar episodes, which after identification are flagged or otherwise denoted on a super-invoice.

In another aspect, the payment system is configured to collect more or one payment to pay an amount owed to one or more service provider. In one aspect where the consumer has sufficient funds to pay, payment is processed using a payment mechanism of the payment system. In another aspect where the consumer does not have sufficient funds to pay, the payment system is configured to establish a funding campaign to collet commitments to pay all or a portion of an amount owed, which in some aspects is all or part of an amount presented in a super-invoice. In another aspect, the funding campaign is broadcast, shared, and/or disseminated by the consumer and/or one or benefactors and/or patrons using a distribution mechanism of the payment system such that the funding campaign is presented on one or more social media and/or network platforms, e.g., Facebook, Twitter, instant messengers, email platforms, and the like. In an aspect of the present disclosure, the funding campaign concludes with all or a portion of the amount owed being committed to payment by one or more benefactors and payment is allocated to one or more service providers using a payment mechanism of the payment system. In one aspect where the collected funds are insufficient, the payment system is configured to notify the service providers of the consumer affordability score of the consumer based on personal and economic information of the consumer, whereby the consumer affordability score is derived by an algorithm of the payment system to demonstrate the capability of a consumer to pay the amount owed. In some aspects of the presented disclosure, an offer is provided by the payment system to provide a reduced payment to satisfy the amount owed, which can be rejected, countered, or accepted by each or all of one or more service providers.

In one aspect, a payment system is configured to collect, aggregate, and display quality ratings for service provider or a product.

This has outlined, rather broadly, the features, advantages, solutions, and benefits of the disclosure in order that the description that follows may be better understood. Additional features, advantages, solutions, and benefits of the disclosure will be described in the following. It should be appreciated by those skilled in the art that this disclosure may be readily utilized as a basis for modifying or designing other structures and related operations for carrying out the same purposes of the present disclosure. It should also be realized by those skilled in the art that such equivalent constructions and related operation do not depart from the teachings of the disclosure as set forth in the appended claims. The novel features, together with further objects and advantages, will be better understood from the following description when considered in connection with the accompanying Figures. It is to be expressly understood, however, that each of the Figures is provided for the purpose of illustration and description only and is not intended as a definition of the limits of the present disclosure.

### BRIEF DESCRIPTION OF THE DRAWINGS

FIG. 1 is a diagram of a method and system for social payment of medical bills and medical settlement according to an aspect of the disclosure;

FIG. 2 is a flowchart of a method and system for social payment of medical bills and medical settlement according to an aspect of the disclosure;

FIG. 3 is a flowchart of a method and system for social payment of medical bills and medical settlement according to an aspect of the disclosure;

FIG. 4 is a flowchart of a method and system for social payment of medical bills and medical settlement according to an aspect of the disclosure;

FIG. 5 is a flowchart of a method and system for social payment of medical bills and medical settlement according to an aspect of the disclosure;

FIG. 6 is a flowchart of a method and system for social payment of medical bills and medical settlement according to an aspect of the disclosure;

FIG. 7A is a flowchart of a method and system for social payment of medical bills and medical settlement according to an aspect of the disclosure;

FIG. 7B is a flowchart of a method and system for social payment of medical bills and medical settlement according to an aspect of the disclosure;

FIG. 8A is a flowchart of a method and system for social payment of medical bills and medical settlement according to an aspect of the disclosure;

FIG. 8B is a flowchart of a method and system for social payment of medical bills and medical settlement according to an aspect of the disclosure;

FIG. 8C is a flowchart of a method and system for social payment of medical bills and medical settlement according to an aspect of the disclosure;

FIG. 8D is a flowchart of a method and system for social payment of medical bills and medical settlement according to an aspect of the disclosure;

FIG. 8E is a flowchart of a method and system for social payment of medical bills and medical settlement according to an aspect of the disclosure;

FIG. 8F is a flowchart of a method and system for social payment of medical bills and medical settlement according to an aspect of the disclosure;

FIG. 8G is a flowchart of a method and system for social payment of medical bills and medical settlement according to an aspect of the disclosure;

FIG. 9A is a flowchart of a method and system for social payment of medical bills and medical settlement according to an aspect of the disclosure;

FIG. 9B is a flowchart of a method and system for social payment of medical bills and medical settlement according to an aspect of the disclosure;

FIG. 9C is a flowchart of a method and system for social payment of medical bills and medical settlement according to an aspect of the disclosure; and

FIG. 10 is a flowchart of a method and system for social payment of medical bills and medical settlement according to an aspect of the disclosure.

### DETAILED DESCRIPTION

The disclosure described herein is directed to different aspects of a method and system for social payment of medical bills and medical settlement. The detailed description set forth below, in connection with the appended drawings, is intended as a description of various configurations and is not intended to represent the only configurations in which the concepts described herein may be practiced. These descriptions include specific details for the purpose of providing a thorough understanding of the various concepts. It will be apparent, however, to those skilled in the art that these concepts may be practiced without these specific details. In some instances, well-known structures and components are shown in block diagram form in order to avoid obscuring such concepts. As described herein, the use of the term “and/or” is intended to represent an “inclusive OR”, and the use of the term “or” is intended to represent an “exclusive OR”.

The disclosure is described herein with reference to certain aspects, iterations, embodiments, and examples but it is understood that the disclosure can be embodied in many different forms and should not be construed as limited to the aspects set forth herein. In particular, the disclosure is described herein in regards to a method and system for social payment of medical bills and medical settlement in a healthcare environment, but it is understood that the disclosure can settle balances related to any form of consumer transaction, including those found in e-commerce or any commercial or transactional environment.

It is understood that actual systems or fixtures embodying the disclosure can be arranged in many different ways with many more features and elements beyond what is shown in the drawings. For the same or similar elements or features, the same reference numbers may be used throughout the disclosure.

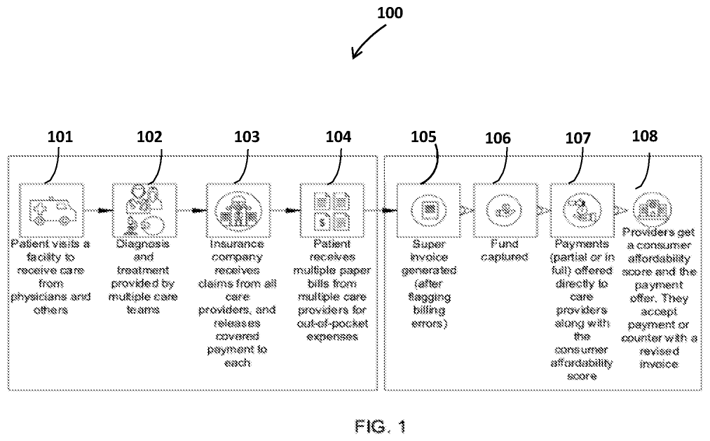

With reference to the Figures, aspects of a method and system for social payment of medical bills and medical settlement with a service provider using a payment system 100, such as a healthcare provider is described. With reference to FIG. 1, a healthcare consumer visits a service provider and avails the medical facilities of the service provider at 101. Next, at 102 based on the health complications and medical needs, the service provider provides medical treatment and necessary medical diagnosis, medical care, and treatment to the healthcare consumer. The medical diagnosis, medical care, and treatment offered by the service provider include all diagnostic and medical test, imaging techniques, medicines, expert opinion, and any other hospital facilities utilized during the medical treatment. A medical claim bill based on the hospital chargemaster is generated and shared with the health insurance provider for reimbursement of the medical expenses covered under the medical insurance of the consumer at 103. An additional medical bill is generated for expenses to be paid by the consumer (out-of-pocket expenses) at 104. A consumer can receive multiple medical bills (104, 104′, 104″) from multiple visits to one or more service providers (101, 101′, 101″).

At 105, a super-invoice is generated from the medical bills provided at 104, 104′, 104″ uploaded or transferred to the payment system. In an aspect of the present disclosure, at 105 after the medical bills are uploaded by the consumer using a smart device or web-based application or the like, the payment system extracts the data from the medical bills using OCR and artificial intelligence (“AI”) technologies to generate the super-invoice 105. Errors in the medical bill due to wrong use of medical code(s) and charges for diagnostic or medical procedures not performed but wrongly entered into the medical claim are flagged before generating the super-invoice. The super-invoice provides a final amount after eliminating all the medical billing errors. In some aspects of the disclosure, at 106 the funds to pay the final amount are captured by one or more avenue described further herein. At 107 the healthcare consumer offers to make full or partial payment to the service provider or providers. In some configurations, a consumer affordability score is generated at 108 for the consumer which is an indication of the capability of the consumer to pay the medical bill or super-invoice.

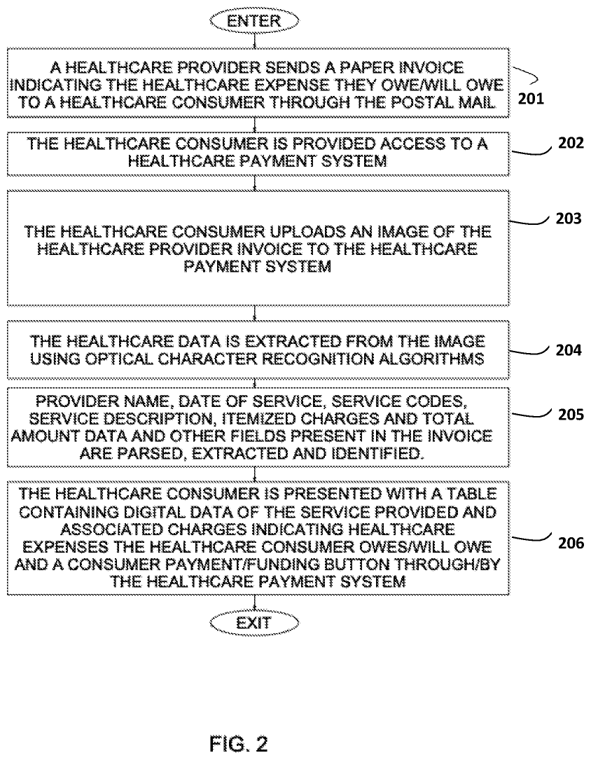

Referring to FIG. 2, the process to upload one or more paper medical bills or invoices received through postal mail or other physical transfer and delivery is described. At 201 a service provider sends the medical bill in tangible form, such as paper, to the healthcare consumer. The healthcare consumer gets access to a healthcare payment system at 202. Then at 203, the healthcare consumer uploads the paper medical bill on the healthcare payment system by uploading an image of the paper medical bill or bills. Next, the healthcare payment system utilizes optical character recognition (“OCR”) algorithms at 204 to analyze, extract, and identify data at 205 such as name of the service provider, date of service, service codes, service description, itemized charges, total amount, and other information and fields present in the medical bill or bills from the image or images and generate a super-invoice at 206 containing healthcare charges and total healthcare expense the healthcare consumer owes to the service provider or service providers.

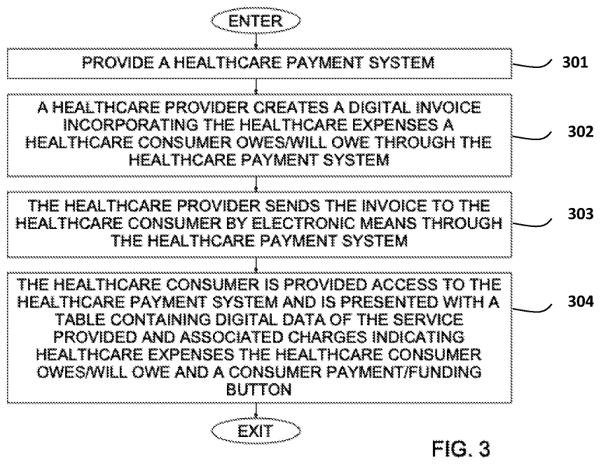

Referring to FIG. 3, the process to upload the medical bill or medical bills received through electronic means is described. The healthcare payment system is provided at 301 and at 302 is accessed by the service provider to create a digital invoice containing healthcare expenses. At 303, the digital invoice is sent to the healthcare consumer via electronic means, such as email, online posting, website submissions, internet transmissions, through the healthcare payment system, or the like. The digital invoice is accessible to the healthcare consumer via the healthcare payment system at 304. Also at 304, a table or super-invoice containing digital data of all the medical expenses and total charges owed is presented to the healthcare consumer for their consideration towards the payment of the medical invoice.

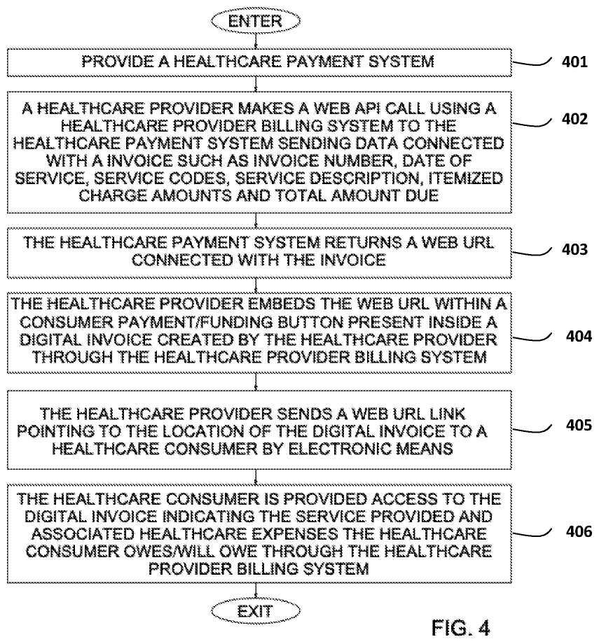

Referring to FIG. 4, the digital process to share the medical bill using a unique uniform resource location (“URL”) by the healthcare service provider with the healthcare consumer is described. The healthcare payment system is provided at 401 and is accessed by the service provider to make a Web API call using the healthcare payment system by entering the data connected with a specific invoice such as invoice number, date of service, service codes, service description, itemized charge amounts, and the total medical bill amount due at 402. The healthcare payment system generates a URL for the specific invoice number at 403 and details entered by the healthcare service provider. The URL generated at 403 is now ready to be shared with the healthcare consumer via a payment and/or funding button or the like provided inside the digital invoice at 404 that is associated with the invoice number at 402. In one aspect, at 405, the URL points to a fixed location on the digital invoice and is sent to the healthcare consumer via electronic means. The healthcare consumer can open the URL by clicking on the URL to open the invoice attached to the URL to access the digital invoice containing the healthcare services availed by the healthcare consumer and the total healthcare expense owed and/or that will be owed by the healthcare consumer at 406.

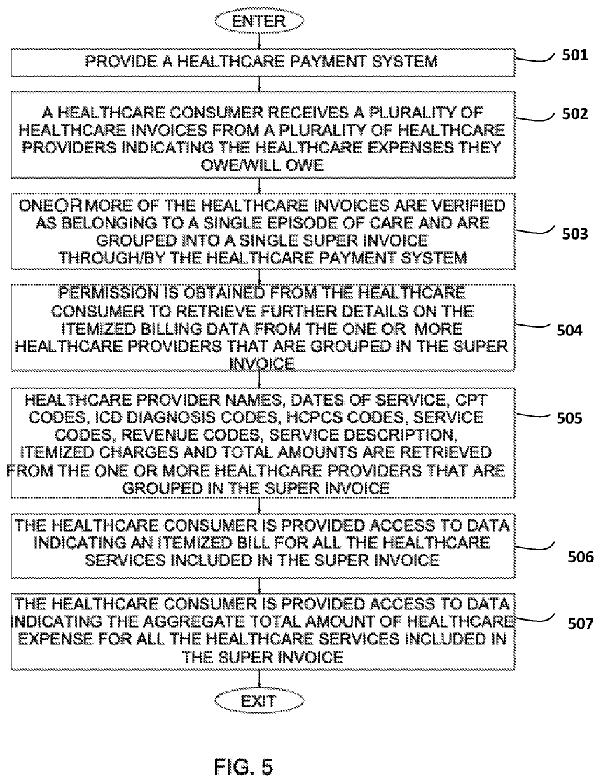

Referring to FIG. 5, a process to generate a super-invoice from a plurality of medical invoices by a plurality of healthcare service providers is described. At 501 the healthcare payment system is provided and the healthcare consumer uploads all the medical invoices received from a plurality of service providers in the healthcare payment system at 502. After a verification step at 503, the healthcare payment system generates a single super-invoice, which in some aspects relates to a single episode of healthcare. The healthcare consumer gives an authorization at 504 to the healthcare payment system to retrieve the details of the itemized billing data from a plurality of healthcare service providers. At 505, data such as the name of the healthcare service provider, date of service, CPT codes, ICD diagnosis, HCPCS codes, service codes, revenue codes, service description, charges for various items, and final total amount due to each healthcare service provider is retrieved and arranged in the single super invoice. A complete itemized bill is generated at 506 and the access to complete data indicating the charges for healthcare services is provided to the healthcare consumer. Finally, at 507, the healthcare consumer can see the total amount due in agreement to one or more service providers in the super invoice.

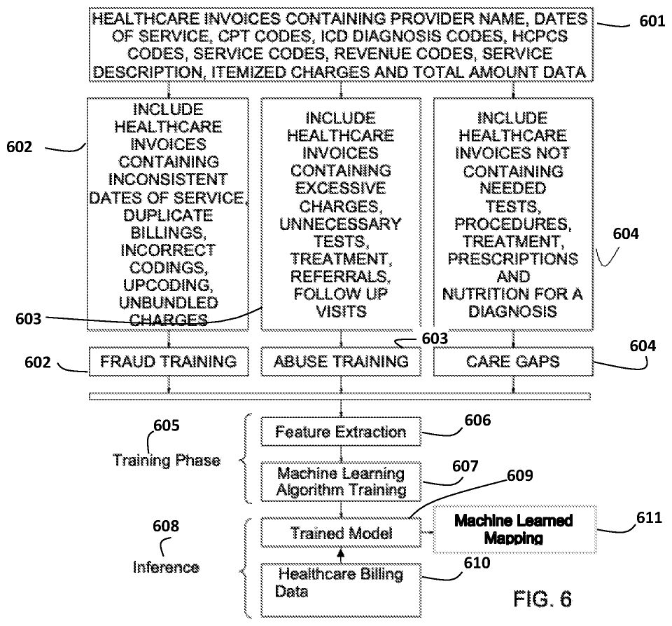

Referring to FIG. 6, the aspects of the healthcare payment system in predicting billing errors and care gaps are described. At 601, one or more healthcare invoices containing service provider names, dates of service, CPT codes, ICD diagnosis codes, HCPCS codes, service codes, revenue codes, service description, itemized charges, and total bill amount data are used as the raw data set for training a machine learning model. A subset of the invoices forming the training dataset contain errors, which include one or more those errors at 602 for fraud training related with inconsistent dates of services, duplicate billings, use of incorrect codes, upcoding, and unbundled charges, and those errors at 603 for abuse training related to excessive charges, unnecessary tests, treatment, referrals, and follow up visits, and those errors at 604 for care gaps related to missing diagnostics tests, procedures, treatment prescriptions, and essential nutritional advisory needed for the treatment and management of the medical condition identified by the ICD codes in the bills.

The training phase at 605 comprises feature extraction at 606 in some aspects and machine learning and algorithm training at 607 in some aspects. At 606, feature extraction comprises the selection or combination of key variables within the raw data contained in the medical invoices to reduce the amount of data that must be processed, without losing important or relevant information from the original data set. The feature extraction at 606 facilitates the speed of leaning and generalization steps in the machine learning process 607. In one aspect, in the learning phase at 607 comprises supervised, unsupervised, or hybrid learning techniques to train a model to predict fraud, abuse, and care gaps within any given medical invoice. Once an acceptable billing error detection performance is obtained the training phase at 605 is complete and the inference phase at 608 begins. During the inference phase the trained model is deployed at 609e. The healthcare consumer uploads medical bills at 610 that were received from a service provider into the healthcare payment system. The contents of the medical bills are passed through the trained model at 609 to predict billing errors and gaps in care in real-time based on machine learned mapping at 611.

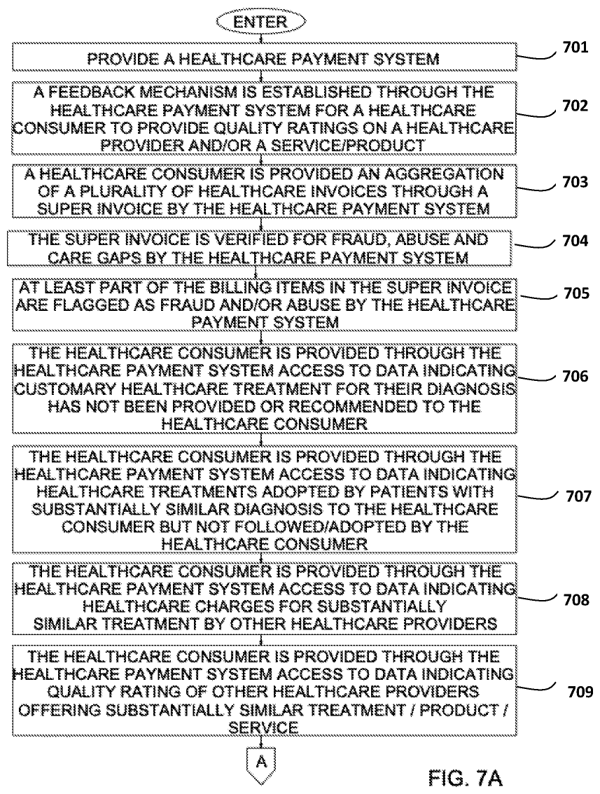

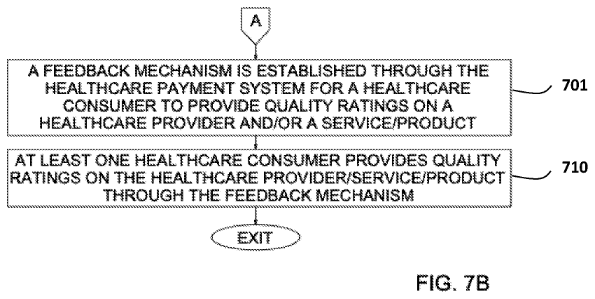

Referring to FIGS. 7A & 7B, the healthcare payment system is provided at 701 and at 702, the healthcare payment system provides a feedback mechanism for a healthcare consumer to rate the healthcare services offered by the service provider. The feedback mechanism helps potential future healthcare consumers in accessing the quality of the healthcare services and/or products offered by the service provider. The healthcare payment system generates a super invoice at 703 containing an aggregate bill of all the healthcare services availed by the healthcare consumer for a single episode of care from a plurality of service providers. The super invoice is verified via a verification step at 704 to eliminate errors in the super invoice by highlighting fraud, abuse, or care gaps as detailed herein. During the verification step, some of the errors in the super invoice may be flagged as fraud and/or abuse by the healthcare payment system at 705. The healthcare payment system provides access to a database at 706 indicating customary healthcare treatment options not provided or recommended to the healthcare consumer. In one aspect, another database provides information regarding treatment options used by other healthcare consumers for substantially similar diagnosis but not adopted by the healthcare consumer at 707. In one aspect, at 708 another database contains data about the healthcare charges offered by other service providers for substantially similar treatment, products, and/or services. Another database is provided at 709 in some aspects that contains data of quality ranking of other service providers offering substantially similar treatment, products, and/or services. At 710 at least one healthcare consumer who has availed the service or bought the product of the service provider voluntarily offers the feedback using the feedback mechanism.

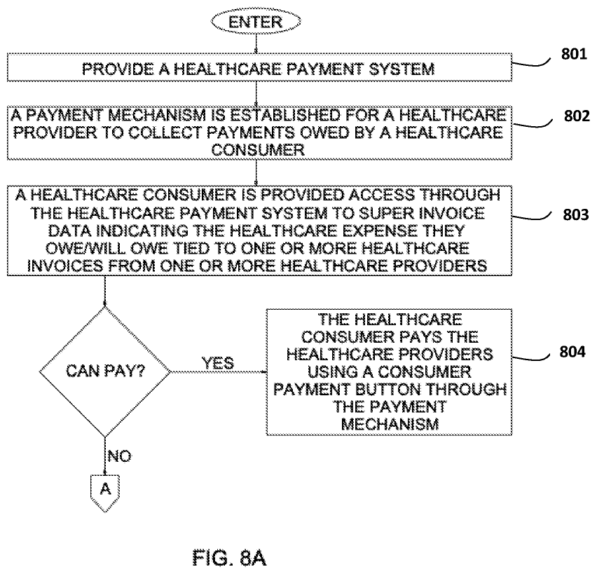

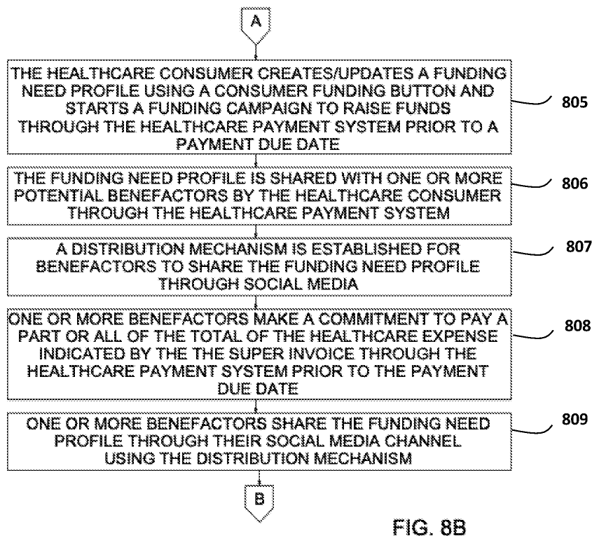

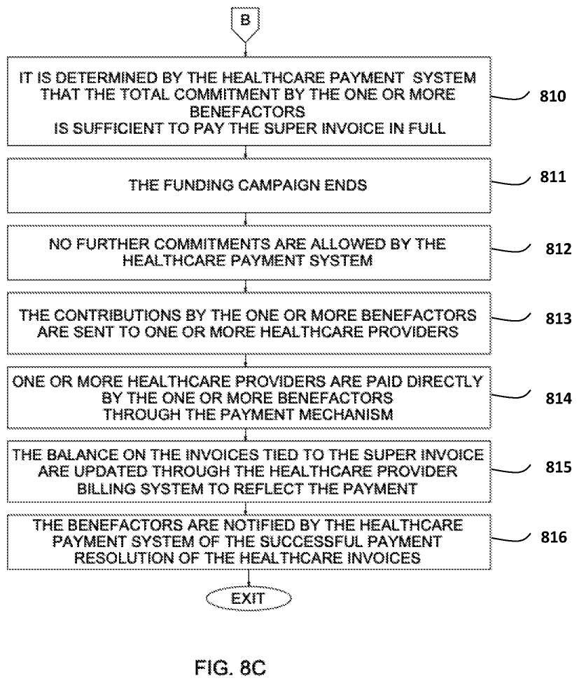

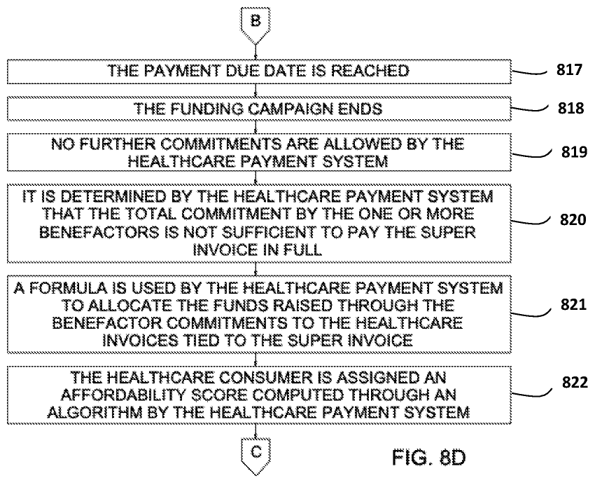

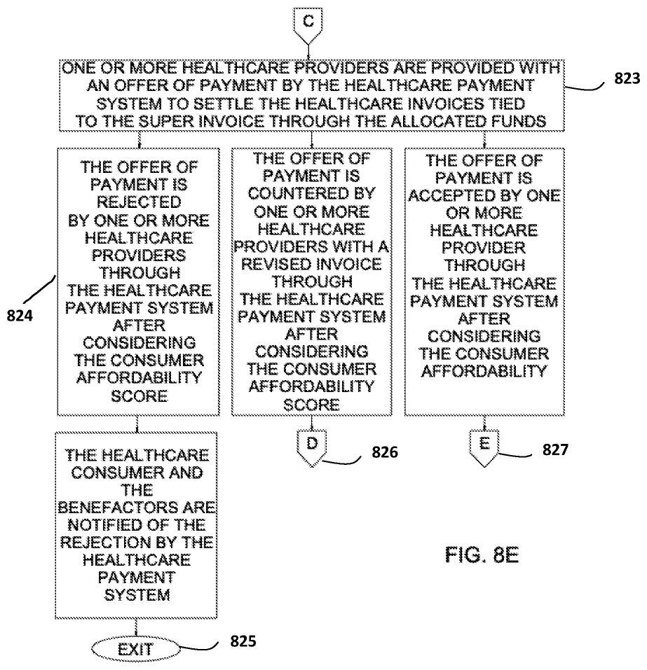

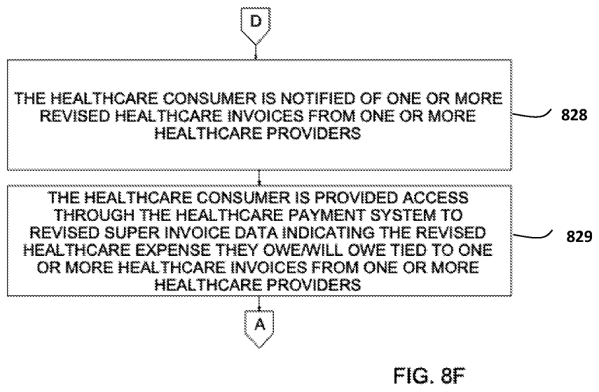

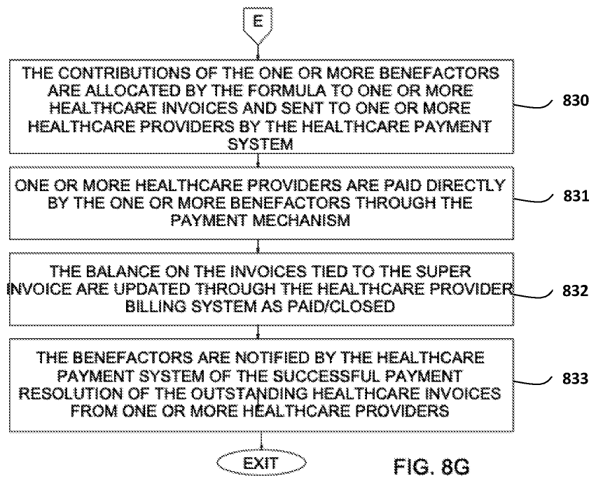

Referring to FIG. 8A, a process to settle the healthcare bill with the service provider is described. At 801, a healthcare payment system is provided. A payment mechanism is established at 802 for the service provider which collects the payment from the healthcare consumer for application to one or more bill, which in some aspects is represented in the super invoice. The healthcare consumer gets access, including by way of the super invoice at 803 indicating the final charges owed to each service provider. The healthcare consumer can directly pay the charges to each healthcare service provider using a payment button provided in the healthcare payment system at 804. However, in cases where the consumer cannot pay the bill due to financial constraints, they can utilize the aspects of the present invention.

Referring to FIG. 8B, a process to raise the funds using the healthcare payment system is described. The healthcare consumer creates a fundraiser by inputting a funding need profile at 805 before the due date of the bill using the healthcare payment system. The fundraiser is shared with friends and family to seek financial contributions at 806. A distribution mechanism is established at 807 that allows friends and family to further distribute the fundraiser to their social media and/or network platforms and the like, which helps in reaching more potential financial contributors. The benefactors make a commitment to pay the healthcare expenses at 808, fully or partially, before the due date of the super invoice created at 803. The benefactors share the fundraiser through social media and/or network platforms and the like with other potential benefactors at 809. Benefactors may include family, friends, other patrons of the healthcare consumer; and/or entities who are not directly or personally connected to the healthcare consumer.

Referring to FIG. 8C, the process to close the fundraiser is described. At 810 the healthcare payment system determines if the total funds committed by one or more of the benefactors are sufficient to pay the healthcare expenses, whereupon the fundraiser ends at 811 and the healthcare payment system makes the decision at 812 to close any future commitments towards the super invoice. The contributions committed by the benefactors are notified to the services providers at 813. One or more service providers are paid directly by the one or more contributors at step 814. The balance on the pending invoices are updated at 815 to reflect complete payment of the obligations by the healthcare payment system and the benefactors are notified via a notification at 816 of a successful payment transaction.

Referring to FIG. 8D, the process to settle the healthcare expenses is described when the funds collected up to the payment due date via the fundraiser are not sufficient to pay the total amount of the super invoice. At 817, the fundraiser ends on the payment due date and the healthcare payment system ends the fundraising campaign at 818. At 819, the healthcare payment system determines whether to stop any future commitments towards payment of the pending bills. In one aspect, if the total funds committed by one or more of the benefactors are not sufficient to pay the healthcare expenses at 820, then the healthcare payment system uses a formula to allocate the raised funds to the service provider's owed money in the super invoice at 821. In some aspects, the healthcare consumer is assigned a consumer affordability score using an algorithm that uses the personal and social information, including the size of the consumer's online social media and/or network, of the healthcare consumer to derive the consumer affordability score at 822.

Referring the FIG. 8E, a process to settle the healthcare expense bill in the case of insufficient funds is described. In circumstances where the funds raised through a fundraiser are not sufficient to pay the healthcare expense in full, a payment offer is made to one or more service providers at 823 based on the allocated funds at 821. In one aspect, one or more service providers can reject the payment offer at 824 and the benefactors and healthcare consumer are informed of the rejection decision by a provider through a notification at 825. In another aspect, one or more service providers can negotiate a revision of the invoice at 826 by reducing healthcare expenses owed to an acceptable amount after considering the consumer affordability score derived at 822. In another aspect, one or more service providers can accept the payment offer proposed by the healthcare payment system at 827 after consideration of the consumer affordability score.

Referring to FIG. 8F, when one or more service providers issue a revised invoice after considering the consumer affordability score at 822, the healthcare consumer is notified via a notification at 828. The healthcare consumer, in some configurations, can raise additional funds from one or more benefactors to fulfill the revised or invoices at 829.

Referring to FIG. 8G, when a settlement offer proposed by the healthcare payment system is accepted by one or more healthcare service providers, the contributions received from one or more benefactors are allocated at 830 to one or more service providers using the formula at 821. Once allocated, payment to the service providers is made directly by one or more benefactors at 831. After payment, the final pending amount is updated and the one or more bills from one or more service providers is marked as paid and/or closed at 832. One or more benefactors are informed via notification by the healthcare payment system of a successful closure of one or more pending bills at 833.

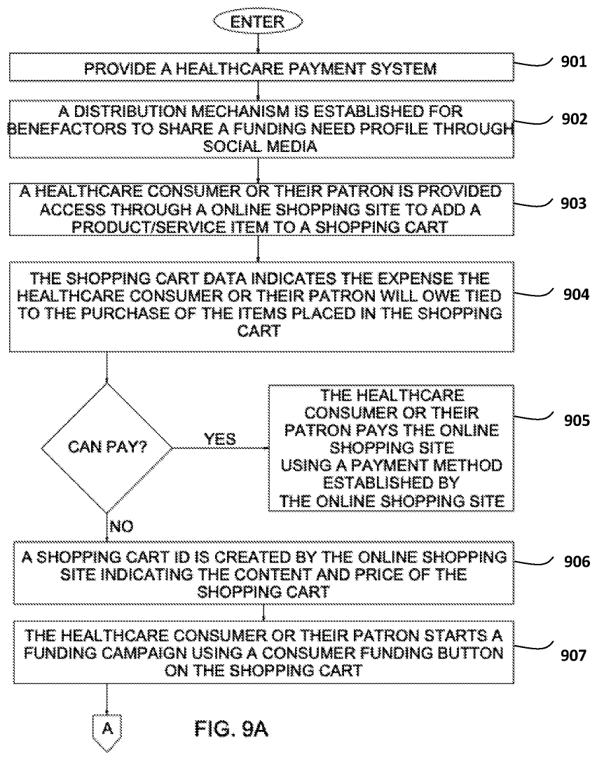

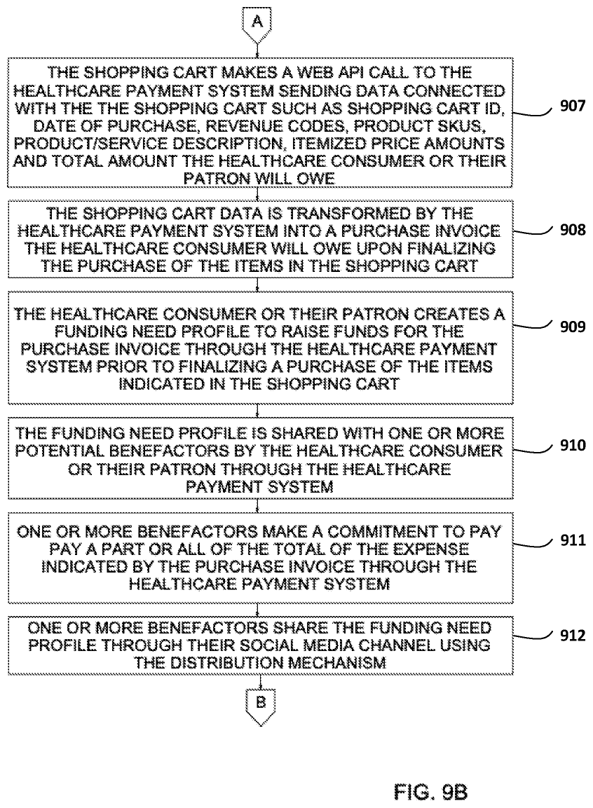

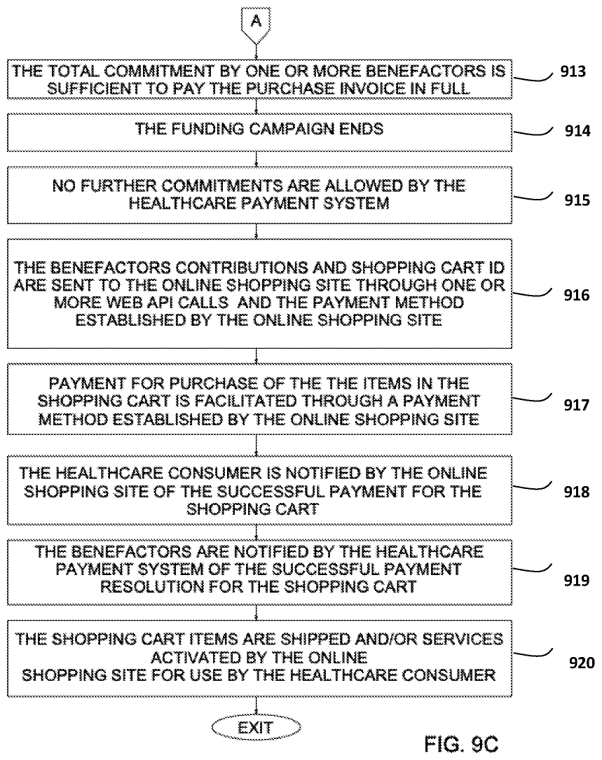

Referring to FIG. 9A, a process to share an online purchase of products and services with friends, family, and patrons seeking contributions towards payment for fulfilling an online order is described. The healthcare payment system is provided at 901 and at 902 the healthcare payment system provides a distribution mechanism for benefactors to share an online order on social media and/or networks and the like to generate funds for the purchase of healthcare services and products via online retailers. A healthcare consumer or their patron accesses an online retailer's website or other platform and adds desired services or products into a shopping cart at 903. After items and/or services are added to the shopping cart, then at 904 the shopping cart displays the final amount the consumer owes and/or will owe to complete the purchase.

If the healthcare consumer or their patron are able to pay the online order in full, then at 905 the full payment is instantly made by the healthcare consumer or their patron and the order is fulfilled with a product shipped or an item of service made available. Alternatively, if the healthcare consumer or their patron cannot pay for the online order in full, then at 906 a shopping cart identification or ID is generated indicating the total products and services content and the net price owed by the consumer. The healthcare consumer or their patron can then initiate one or more fundraiser 907, which in one aspect is completed by using a consumer funding option, “Fund This Bill”, available on an online order payment form. Referring to FIG. 9B, the shopping cart provided by the online retailer makes a Web API call to the healthcare payment system to send data related with the purchase being made by the healthcare consumer at 907. This data includes, but is not limited to, shopping cart ID, date of purchase, revenue codes, product SKUs, product description, itemized price amount, and/or the total amount the healthcare consumer or their patron will owe to fulfill the pending online order for products and/or services. At 908, the healthcare payment system converts the data of the shopping cart into a purchase order containing the total amount the healthcare consumer will owe. Next, the healthcare consumer or their patron starts a fundraiser to generate funds at 909 for the purchase of the healthcare services or products. The funding need profile is shared at 910 with one or more benefactors for a contribution towards purchasing the services and products from the online shopping platform. At 911, one or more benefactors make a commitment to pay in part or in full for the purchase order through the healthcare payment system. Additionally, one or more benefactors can share the purchase order through their social media and/or network at 912 using the distribution mechanism to reach more potential benefactors.

Referring to FIG. 9C, at 913 the total amount committed by one or more benefactors is sufficient to pay for the purchase order, prompting the fundraising to end at 914 whereupon the healthcare payment system stops accepting further commitments at 915. The contribution commitments from the benefactors and the shopping cart ID are sent to the online retailer at 916. Payment is processed through one or more payment methods established by and/or with the online retailer to pay the amount for the purchase at 917. The consumers are notified by the healthcare payment system after successful fulfillment of the online order at 918 as are the one or more benefactors at 919. Thereafter, at 920, the purchase products and services are shipped or made available for use to the consumer. Therefore, a method and system for social payment of medical bills and medical settlement, including aggregate online purchases that are recurring as well as pre-service and post-service payment of one or more services in the aggregate, has been provided that allows a consumer to upload one or more bills received from one or more service providers, including healthcare service providers; utilizes big data, artificial intelligence, and deep learning technologies to extract and identify billing errors, including incorrect pricing, coding errors, and the like; provides a comparison of service prices offered by other service providers in the vicinity; generates a super-invoice by aggregating multiple bills, including when multiple service providers are involved; provides direct online payment, partial or complete, by the consumer, friends, family, and patrons thereof; shares a super-invoice with friends, family, and patrons for a contribution towards the payment of the super-invoice; generates e a consumer affordability score based on personal and social information of the consumer which is an indicator of the ability of the consumer to pay; utilizes artificial intelligence and deep learning to rectify billing errors, inconsistencies, and overcharges; maximizes recovery of amounts owed by consumers without the need for repeated manual follow-ups, bill collection actions, or litigation; guides service providers to accept or reject a proposed settlement offer based on the consumer affordability score; identifies items of needed care not provided or sought by the consumer for a specific diagnosis; and improves upon the art.

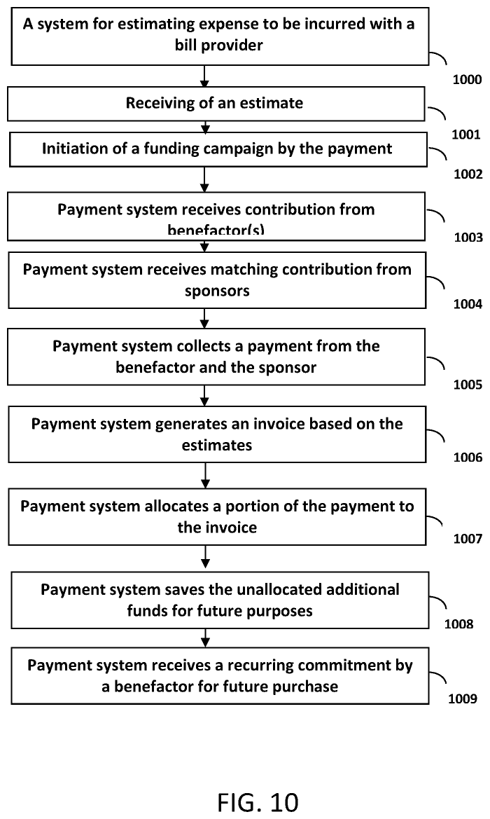

Referring to FIG. 10, a payment system that receives an estimate of bills from the bill provider and initiates a funding campaign is disclosed. A payment system configured to estimate a future expense to be incurred by a consumer is provided at 1000. The payment system receives estimate of an upcoming bill from a bill provider at 1001. The payment initiates a funding campaign and shares the funding campaign on various social media/network platforms by way of the distribution described herein at 1002. At 1003, the payment system accepts contributions towards payment of the estimated bill from one or more benefactors. At 1004, in one aspect the payment system is receives matching contribution from one or more sponsor, which in some aspects is compulsory, and subsequently collects the contributions from the one or more benefactors and the one or more sponsors at 1005. Based on the bill estimates, the payment system generates an invoice at 1006 and allocates the collected contributions towards the settlement of the raised invoice at 1007. In one aspect, the payment system saves the unallocated contributions towards payment of one or more future invoice at 1008 including an amount owed to the service provider or collects the contributions from the benefactors on a recurring basis at 1009.

From the above discussion and accompanying figures and claims it will be appreciated that the method and system for social payment of medical bills and medical settlement offers many advantages over the prior art. Although the present disclosure and its advantages have been described in detail, it should be understood that various changes, substitutions, modifications, and alterations can be made herein without departing from the technology of the disclosure as defined by the appended claims. The scope of the present application is not intended to be limited to the particular configurations of the process, machine, manufacture, composition of matter, means, methods and steps described in the specification only expressly stated otherwise. As one of ordinary skill in the art will readily appreciate from the disclosure, processes, machines, manufacture, compositions of matter, means, methods, or steps, presently existing or later to be developed that perform substantially the same function or achieve substantially the same result as the corresponding configurations described herein may be utilized according to the present disclosure. Accordingly, the appended claims are intended to include within their scope such processes, machines, manufacture, compositions of matter, means, methods, or steps.

The previous description of the disclosure is provided to enable any person skilled in the art to make or use the disclosure. Various modifications to the disclosure will be readily apparent to those skilled in the art, and the generic principles defined herein may be applied to other variations without departing from the spirit or scope of the disclosure. Thus, the disclosure is not intended to be limited to the examples and designs described herein but is to be accorded the widest scope consistent with the principles and novel features disclosed herein

Those of skill would further appreciate that the various illustrative logical blocks, modules, circuits, and algorithm steps described in connection with the disclosure herein may be implemented as electronic hardware, computer software, or combinations of both. To clearly illustrate this interchangeability of hardware and software, various illustrative components, blocks, modules, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The various illustrative logical blocks, and modules described in connection with the disclosure herein may be implemented or performed with a general-purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, multiple microprocessors, one or more microprocessors in conjunction with a DSP core, or any other such configuration.

The steps of a method or algorithm described in connection with the disclosure may be embodied directly in hardware, in a software module executed by a processor, or in a combination of the two. A software module may reside in RAM, flash memory, ROM, EPROM, EEPROM, registers, hard disk, a removable disk, a CD-ROM, solid state storage, or any other form of storage medium known in the art. An exemplary storage medium is coupled to the processor such that the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal. In yet other aspects, the processor can be remote to the storage medium and accesses the storage medium through a linked connection.

In one or more exemplary designs, the functions described may be implemented in hardware, software, firmware, or any combination thereof. If implemented in software, the functions may be stored on or transmitted over as one or more instructions or code on a computer-readable medium. Computer-readable media includes both computer storage media and communication media including any medium that facilitates transfer of a computer program from one place to another. A storage media may be any available media that can be accessed by a general purpose or special purpose computer. By way of example, and not limitation, such computer-readable media can include RAM, ROM, EEPROM, CD-ROM or other optical disk storage, magnetic disk storage or other magnetic storage devices, solid state, or any other medium that can be used to carry or store specified program code means in the form of instructions or data structures and that can be accessed by a general-purpose or special-purpose computer, or a general-purpose or special purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes compact disc (CD), laser disc, optical disc, digital versatile disc (DVD), floppy disk and Blu-ray disc where disks usually reproduce data magnetically, while discs reproduce data optically with lasers. Combinations of the above should also be included within the scope of computer-readable media.

In the present disclosure, the processor may serve as a structure for computer-implemented functions as described herein because the function(s) described in one or more aspects of the present disclosure are coextensive with the processor itself. Further, such a processor may serve as structure for functions that may be achieved by a general purpose computer without special programming, because the coextensive functions include receiving data, storing data, processing data, etc. Further, the present disclosure are removed from the abstract, and do not merely limit the use of an abstract idea to a particular technological environment. The present disclosure expands basic building blocks beyond the mere sum of the parts, at least for the reason that the present disclosure provides faster, more consistent, and more reliable results than obtainable with current methods and devices.

What is claimed is:

1. A system for bill settlement comprising:
a payment system configured to receive a plurality of invoices and extract data from the plurality of invoices;
wherein the payment system is configured to provide a super-invoice that includes the data from the plurality of invoices and an aggregate amount owed;
wherein the payment system is configured to create a funding campaign;
the payment system having a distribution mechanism that is configured to transmit the funding campaign to a social network;
wherein the payment system is configured to receive a commitment by a benefactor to provide a payment to the super-invoice;
wherein the payment system is configured to determine whether the commitment is sufficient to pay the aggregate amount owed of the super-invoice;
the payment system having a payment mechanism to collect a payment from the benefactor based on the commitment; and
the payment system having a formula to allocate the payment to the plurality of invoices directly from the payment system.

2. The system of claim 1 further comprising the payment system having a trained model to identify at least one error, wherein the at least one error is selected from a group consisting of a billing error, a billing abuse, and a billing gap in the data from the plurality of invoices.

3. The system of claim 2 wherein the payment system is configured to provide an offer of payment to settle a portion of the super-invoice based on the error identified by the trained model.

4. The system of claim 1 wherein the payment system is configured to verify that the plurality of invoices belong to a single episode.

5. The system of claim 1 further comprising the payment system having a feedback mechanism configured to receive a quality rating for a service provider and provide quality ratings of other service providers.

6. The system of claim 1 further comprising the payment system having a feedback mechanism configured to receive a quality rating for a product and provide quality ratings of other products.

7. The system of claim 1 wherein the payment system is configured to compute and assign an affordability score to a consumer.

8. The system of claim 7 wherein the payment system is configured to provide an offer of payment to settle a portion of the super-invoice based on the affordability score of the consumer.

9. The system of claim 1 wherein the plurality of invoices are medical invoices.

10. The system of claim 9 wherein the plurality of invoices include medical services that have not been rendered.

11. The system of claim 1 wherein the plurality of invoices include medical products that have not been provided.

12. The system of claim 1 wherein the plurality of invoices are online retailer invoices for completing an online order.

13. The system of claim 1 wherein the payment system is configured to extract the data using optical character recognition.

14. The system of claim 1 further wherein the trained model, during a training phase, is configured to use machine learning for fraud training in order to identify inconsistent dates of service, duplicate billing entries, incorrect service codes, incorrect product codes, and unbundled charges.

15. The system of claim 1 further wherein the trained model, during a training phase, is configured to use machine learning for abuse training in order to identify excess charges, unnecessary products, and unnecessary services.

16. The system of claim 1 wherein the trained model, during a training phase, is configured to use machine learning to identify care gaps.

17. The system of claim 1 wherein the payment system is configured to generate a shopping cart ID associated with a shopping cart of an online retailer.

18. A system for bill settlement comprising:
a payment system configured to create a funding campaign;
the payment system having a distribution mechanism that is configured to transmit the funding campaign to a social network, wherein the payment system is configured to receive a commitment by a benefactor to provide a payment to a service provider and to receive a matching commitment by a sponsor that matches the commitment of the benefactor; and
the payment system having a payment mechanism configured to collect a payment from the benefactor and the sponsor based on the commitment and the matching commitment.

19. The system of claim 18 wherein the estimate of expense from a bill provider is converted into an invoice that includes amount owed to the service provider.

20. The system of claim 18 wherein unallocated excess of the payment received is used for payment of a future invoice.
